SEQUENCE LISTING (4 sequences)

SEQ ID NO: 1 - protein, 293 aa, Homo sapiens [sequence omitted]

SEQ ID NO: 2 - protein, 158 aa, HOMO SAPIENS
GHVGLGVKCSKEVATAIRGAIILAKLIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFTDHLVKTHTRVSVQRTQAPAVATT

SEQ ID NO: 3 - protein, 77 aa, HOMO SAPIENS
HMSIVPVRRGYWGNKIGKPHTVPCKVTGRCGSVLVRLIPAPRGTGIVSAPVPKKLLMMAGIDDCYTSARGCTATLGN

SEQ ID NO: 4 - protein, 87 aa, HOMO SAPIENS
GHVGLGVKCSKEVATAIRGAIILAKLSIVPVRRGYWGNKIGKPHTVPCKVTGRCGSVLVRLIPAPRGTGIVSAPVPKKLLMMAGIDD

(19) United States
(12) Patent Application Publication
(10) Pub. No.: US 2023/0210942 A1
(43) Pub. Date: Jul. 6, 2023

(54) METHODS AND COMPOSITIONS FOR MODULATING ENDOTHELIAL CELL DYSFUNCTION

(71) Applicant: IMAGINE PHARMA LLC, DEVON, PA (US)

(72) Inventors: NGOC THAI, PITTSBURGH, PA (US); JONATHAN POLLETT, PITTSBURGH, PA (US)

(73) Assignee: IMAGINE PHARMA LLC, DEVON, PA (US)

(21) Appl. No.: 16/960,505

(22) PCT Filed: Jan. 9, 2019
(86) PCT No.: PCT/GB2019/050049

§ 371 Date: Jul. 7, 2020

Related U.S. Application Data
(60) Provisional application No. 62/615,223, filed on Jan. 9, 2018.

(51) Int. Cl. A61K 38/17; A61P 9/12

(52) U.S. Cl. CPC A61K 38/1709; A61P 9/12

(57) ABSTRACT

Provided herein is a polypeptide or formulation comprising a polypeptide for use in the treatment of a disease characterized by endothelial dysfunction, wherein the polypeptide or a fragment thereof comprising an amino acid comprising an amino acid sequence having at least 55% sequence identity to the amino acid sequence set forth in SEQ ID NO: 1, 2, 3, or 4. Also provided are methods and uses relating to the same.

### CROSS REFERENCE TO RELATED APPLICATIONS

This application claims the benefit of earlier filed U.S. Provisional Patent Application No. 62/615,223, filed on Jan. 9, 2018, the contents which are herein incorporated by reference in their entirety.

### BACKGROUND OF THE INVENTION

Alterations of endothelial cells and the vasculature play a central role in the pathogenesis of a wide spectrum of human diseases. (See “The Vascular Endothelium and Human Diseases” Int. J. of Bio. Sciences, 2013; 9(10):1057-1069 for a more comprehensive review) The endothelium, which is comprised of a layer of endothelial cells, lines the interior surface of blood vessels and is involved in critical aspects of the vascular system, such as providing a barrier function, blood clotting, inflammation, formation of new blood vessels, vasoconstriction and vasodilation. The endothelium is directly involved in peripheral vascular disease, stroke, heart disease, diabetes, insulin resistance, chronic kidney failure, tumor growth, metastasis, venous thrombosis, and severe viral infectious diseases. Dysfunction of the vascular endothelium is thus a hallmark of human diseases and impaired endothelial function is often seen in patients with many clinical diseases, including but not limited to: hyperglycemia and diabetes (Type I and Type II) and cardiovascular disease, including stroke, pulmonary hypertension, and peripheral vascular disease, arteriosclerosis, and hypercholesterolemia.

### SUMMARY OF THE INVENTION

The present invention relates to compositions and methods for the treatment of diseases related to endothelial cell dysfunction. It has been discovered that compositions of the present invention are capable of normalizing the growth of dysfunctional endothelial cells isolated from diabetic animals, as well as normalize the growth of dysfunctional endothelial cells isolated from patients suffering from pulmonary hypertension. Compositions of the present invention were also shown to increase the growth rates of endothelial cells compared to control, while decreasing the viability of deleterious pulmonary arterial hypertension (PAH) endothelial cells. Compositions of the present invention are also useful in restoring cellular homeostasis in many cell types, including endothelial cells.

These surprising results indicate the compositions are effective to treat many clinical diseases in mammals, including humans, especially those that affect endothelial cells (diabetes, strokes, peripheral vascular disease, hypertension, and atherosclerosis). The beneficial effects may be related to its augmentation of the blood vessel healing and regeneration by its stimulation and support of endothelial cells and endothelial progenitor cells, as well as a restoration or regulation of cellular homeostasis.

In one embodiment of the present invention comprises a composition, formulation and/or medicament of the present invention comprising an active agent comprising a protein or active polypeptide (whether natural or synthetic, (D or L) and including modifications, fragments and analogs thereof) wherein the polypeptide comprises an amino acid sequence from one or more of SEQ ID NO(s): 01-04, listed below at Table I. SEQ ID NO: 01 represents a fragment comprising an active region of a 40s ribosomal protein S2 (RPS2), while SEQ ID NO(s) 02, 03 and 04 represent fragments thereof.

TABLE I
SEQ ID NO(s): 01-04:

SEQ ID NO: 01 (293 aa; fragment of approx. 31 kDa)
MADDAGAAGGPGGPGGPGMGNRGGFRGGFGSGIRGRGRGRGRGRGRGRGA
RGGKAEDKEWMPVTKLGRLVKDMKIKSLEEIYLFSLPIKESEIIDFFLGA
SLKDEVLKIMPVQKQTRAGQRTRFKAFVAIGDYNGHVGLGVKCSKEVATA
IRGAIILAKLSIVPVRRGYWGNKIGKPHTVPCKVTGRCGSVLVRLIPAPR
GTGIVSAPVPKKLLMMAGIDDCYTSARGCTATLGNFAKATFDAISKTYSY
LTPDLWKETVFTKSPYQEFTDHLVKTHTRVSVQRTQAPAVATT
SEQ ID NO: 2: (159 aa; fragment of approx. 17 kDa)
GHVGLGVKCSKEVATAIRGAIILAKLIVPVRRGYWGNKIGKPHTVPCKVT
GRCGSVLVRLIPAPRGTGIVSAPVPKKLLMMAGIDDCYTSARGCTATLGN
FAKATFDAISKTYSYLTPDLWKETVFTKSPYQEFTDHLVKTHTRVSVQRT
QAPAVATT
SEQ ID NO: 3: (75 aa; fragment of approx. 8 kDa)
SIVPVRRGYWGNKIGKPHTVPCKVTGRCGSVLVRLIPAPRGTGIVSAPVP
KKLLMMAGIDDCYTSARGCTATLGN
SEQ ID NO: 4 (87 aa; fragment of approx. 9 kDa)
GHVGLGVKCSKEVATAIRGAIILAKLSIVPVRRGYWGNKIGKPHTVPCKV
TGRCGSVLVRLIPAPRGTGIVSAPVPKKLLMMAGIDD

In a first aspect of the invention, the polypeptide or polypeptide fragment has at least 55%, 60%, 65%, 70%, 75%, 80%, 85%, 90%, 95%, 98% or 99% sequence identity to the amino acid sequence set forth in SEQ ID NO: 1, 2, 3, 4. In one embodiment, the polypeptide or polypeptide fragment has at least 55%, 60%, 65%, 70%, 75%, 80%, 85%, 90%, 95%, 98% or 99% sequence identity to the amino acid sequence set forth in SEQ ID NO: 1. In one embodiment, the polypeptide or polypeptide fragment has at least 55%, 60%, 65%, 70%, 75%, 80%, 85%, 90%, 95%, 98% or 99% sequence identity to the amino acid sequence set forth in SEQ ID NO: 2. In one embodiment, the polypeptide or polypeptide fragment has at least 55%, 60%, 65%, 70%, 75%, 80%, 85%, 90%, 95%, 98% or 99% sequence identity to the amino acid sequence set forth in SEQ ID NO: 3. In one embodiment, the polypeptide or polypeptide fragment has at least 55%, 60%, 65%, 70%, 75%, 80%, 85%, 90%, 95%, 98% or 99% sequence identity to the amino acid sequence set forth in SEQ ID NO: 4. Examples of formulations and pharmaceutical compositions comprising an active agent wherein the active agent comprises a polypeptide or fragment thereof according to one or more of SEQ ID NO(s) 1-4 are described in United States Patent Publication No. 2018/0133280.

In a second aspect the invention provides a polypeptide or formulation comprising a polypeptide for use in the treatment of a disease characterized by endothelial dysfunction, wherein the polypeptide is a polypeptide or polypeptide fragment disclosed herein. The polypeptide may comprise an amino acid sequence having at least 75% sequence identity to the amino acid sequence set forth in SEQ ID NO: 1, 2, 3, or 4. The polypeptide may consist of an amino acid sequence having at least 75% sequence identity to the amino acid sequence set forth in SEQ ID NO: 1, 2, 3, or 4.

The polypeptide may have at least 80%, 85%, 90%, 95%, 98% or 99% sequence identity to the amino acid sequence set forth in SEQ ID NO: 1, 2, 3, or 4. The polypeptide may have at least 80%, 85%, 90%, 95%, 98% or 99% sequence identity to the amino acid sequence set forth in SEQ ID NO: 1. The polypeptide may have at least 80%, 85%, 90%, 95%, 98% or 99% sequence identity to the amino acid sequence set forth in SEQ ID NO: 2. The polypeptide may have at least 80%, 85%, 90%, 95%, 98% or 99% sequence identity to the amino acid sequence set forth in SEQ ID NO: 3. The polypeptide may have at least 80%, 85%, 90%, 95%, 98% or 99% sequence identity to the amino acid sequence set forth in SEQ ID NO: 4.

The polypeptide may comprise the amino acid sequence set forth in SEQ ID NO: 1, 2, 3, or 4. The polypeptide may comprise the amino acid sequence set forth in SEQ ID NO: 1. The polypeptide may comprise the amino acid sequence set forth in SEQ ID NO: 2. The polypeptide may comprise the amino acid sequence set forth in SEQ ID NO: 3. The polypeptide may comprise the amino acid sequence set forth in SEQ ID NO: 4. The polypeptide may consist of the amino acid sequence set forth in SEQ ID NO: 1, 2, 3, or 4. The polypeptide may consist of the amino acid sequence set forth in SEQ ID NO: 1. The polypeptide may consist of the amino acid sequence set forth in SEQ ID NO: 2. The polypeptide may consist of the amino acid sequence set forth in SEQ ID NO: 3. The polypeptide may consist of the amino acid sequence set forth in SEQ ID NO: 4.

The disease characterized by endothelial dysfunction may be selected from cardiovascular disease, atherosclerosis, obesity and pulmonary arterial hypertension. The disease characterized by endothelial dysfunction may be cardiovascular disease, e.g. atherosclerosis. The disease characterized by endothelial dysfunction may be obesity. The disease characterized by endothelial dysfunction may be pulmonary arterial hypertension.

The formulation may be an oral pharmaceutical formulation. The formulation may be a parenteral pharmaceutical formulation. The formulation may be a topical formulation.

The formulation may comprise one or more pharmaceutically acceptable carrier and/or one or more pharmaceutically acceptable diluent and/or one or more pharmaceutically acceptable excipient. The formulation may comprise one or more pharmaceutically acceptable carrier(s). The formulation may comprise one or more pharmaceutically acceptable diluent(s). The formulation may comprise one or more pharmaceutically acceptable excipient(s).

The formulation may be an aqueous pharmaceutical formulation. The peptide may be present at a concentration of 0.05 to 5 μg/L in the aqueous pharmaceutical formulation. For example, the peptide may be present at a concentration of 0.08 to 3 μg/L or may be present at 0.1 to 1 μg/L. The peptide may be present at a concentration of at least 0.05 μg/L, for example at least 0.7, 0.1 or 0.5 μg/L. The peptide may be present at a concentration of not more than 5 μg/L, for example not more than 4, 3 or 2 μg/L (e.g. not more than 1 μg/L). The aqueous pharmaceutical formulation may comprise a buffer. The buffer may have a pH of from about 7 to about 8, for example the buffer may have a pH of from about 7.2 to about 7.6, e.g. the buffer may have a physiological pH (a pH of about 7.4, e.g. a pH of from 7.3 to 7.5). The buffer may be phosphate buffered saline.

In a third aspect the invention provides a method for modulating endothelial cell growth, the method comprising contacting an endothelial cell with a polypeptide or formulation comprising a polypeptide, wherein the polypeptide or formulation is a polypeptide or formulation as disclosed herein (e.g. a polypeptide or formulation of an aspect or embodiment of the invention). The polypeptide may comprise a polypeptide comprising an amino acid sequence having at least 75% sequence identity to the amino acid sequence set forth in SEQ ID NO: 1, 2, 3, or 4. The contacting may be performed in an aqueous solution. The method may be an in vitro method.

In a fourth aspect the invention provides use of a polypeptide or formulation comprising a polypeptide for modulating endothelial cell growth, wherein the polypeptide or formulation is a polypeptide or formulation as disclosed herein (e.g. a polypeptide or formulation of an aspect or embodiment of the invention). The polypeptide may comprise a polypeptide comprising an amino acid sequence having at least 75% sequence identity to the amino acid sequence set forth in SEQ ID NO: 1, 2, 3, or 4.

In a fifth aspect the invention provides a method for treating a disease characterized by endothelial dysfunction, comprising administering to a subject in need thereof of a therapeutically effective amount of a composition comprising a polypeptide as disclosed herein (e.g. a polypeptide of an aspect or embodiment of the invention). The polypeptide may comprise an amino acid sequence having at least 75% sequence identity to the amino acid sequence set forth in SEQ ID NO: 1, 2, 3, or 4, or an active fragment or analogue thereof. The disease characterized by endothelial dysfunction may be selected from cardiovascular disease, atherosclerosis, obesity and pulmonary arterial hypertension. The disease characterized by endothelial dysfunction may be cardiovascular disease, e.g. atherosclerosis. The disease characterized by endothelial dysfunction may be obesity. The disease characterized by endothelial dysfunction may be pulmonary arterial hypertension.

In a sixth aspect the invention provides use of at least one polypeptide as disclosed herein (e.g. a polypeptide of an aspect or embodiment of the invention) for the manufacture of a medicament for the treatment of a disease characterized by endothelial dysfunction. The polypeptide may comprise an amino acid sequence having at least 75% sequence identity to the amino acid sequence set forth in SEQ ID NO: 1, 2, 3, or 4. The disease characterized by endothelial dysfunction may be selected from cardiovascular disease, atherosclerosis, obesity and pulmonary arterial hypertension. The disease characterized by endothelial dysfunction may be cardiovascular disease, e.g. atherosclerosis. The disease characterized by endothelial dysfunction may be obesity. The disease characterized by endothelial dysfunction may be pulmonary arterial hypertension.

In any of the second, third, fourth, fifth or sixth aspects, the polypeptide or at least one polypeptide may be a polypeptide as defined in the first or second aspect.

In any of the third or fourth aspects, the formulation may be a formulation as defined in the second aspect.

### BRIEF DESCRIPTION OF THE DRAWINGS

FIG. 1 shows that treatment of endothelial cells with the peptide molecules increases the number of endothelial cells, as measured by a cell proliferation assay and shown as relative optical density (OD). IMG-1 depicts the cells treated with full length peptide, wherein the nomenclature IMG-1 refers to SEQ ID NO 1; IMG-2 depicts cells treated with the 17 kDa fragment; and IMG-2×depicts cells treated with the 14 kDa peptide fragment, wherein the nomenclature IMG-2 and IMG-2×refer to the fragment sizes purified following cleavage of the full-length fragment; IMG-2 corresponds to SEQ ID NO 2, and IMG-2×corresponds to a fragment of the N-terminus region of the full length protein.

FIG. 2 shows that treatment of endothelial cells with the peptide molecules increases the number of endothelial progenitor cells, as measured by the percent of CD31/CD133 positive cells in culture. IMG-1 depicts the cells treated with full length peptide wherein the nomenclature IMG-1 refers to SEQ ID NO 1; IMG-2 depicts cells treated with the 17 kDa fragment; and IMG-2×depicts cells treated with the 14 kDa peptide fragment, wherein the nomenclature IMG-2 and IMG-2×refer to the fragment sizes purified following cleavage of the full-length fragment.

FIG. 3 shows the effect of IMG-1 on diabetes-related cardiac complications and hypertension. Treatment of Zucker diabetic fatty rats with IMG-1, whether by oral or intravenous administration, results in normalized blood pressure, as measured by mmHg for both systolic and diastolic blood pressure. IMG-1 depicts full length peptide corresponding to SEQ ID NO 1; the dashed lines depict rats treated by intravenous administration and the dashed and dotted lines depict rats treated by oral administration.

FIG. 4 shows that treatment of endothelial cells collected from (Bcl-2 Negative; i.e., non-pulmonary arterial hypertensive patients) patients undergoing pulmonary catheterization results in an increase of cell growth of Bcl-2 negative pulmonary hypertension endothelial cells, as measured by relative viability of cells. In the graph, IMG-1 depicts full-length peptide (SEQ ID NO 1) and IMG-2 depicts the 17 kDa fragment (SEQ ID NO 2) (purified following cleavage of the full-length protein).

FIG. 5 shows that treatment of endothelial cells collected from Bcl-2 Positive patients undergoing pulmonary catheterization results in an impairment of Bcl-2 positive pulmonary hypertension endothelial cell growth, as measured by relative viability of cells. In the graph, IMG-1 depicts full-length peptide (SEQ ID NO 1) and IMG-2 depicts the 17 kDa fragment (SEQ ID NO 2) (purified following cleavage of the full-length protein).

### DETAILED DESCRIPTION OF THE INVENTION

The following terms are used in this disclosure to describe different aspects of the invention. These terms are used for explanation purposes only and are not intended to limit the scope for any aspect of the invention.

As used herein “active ingredient”, “compound”, and/or “active agent” may be used interchangeably and refer to a protein, polypeptide, peptide fragment, or analogue thereof, and including any modification thereto, having an amino acid sequence selected from one or more of SEQ ID NO. 1; or SEQ ID NO: 2, or SEQ ID NO: 3, SEQ ID NO: 4, or combinations thereof. Exemplary formulations of the present invention comprising the active ingredient/compound may be referred to herein using the nomenclature “IMG-1” or “IMG-2” in reference to formulations, without implying any specific dosage or concentration of active compound.

As used herein “pharmaceutical formulation”, “pharmaceutical composition”, “formulation”, or “composition” refer (interchangeably) to a liquid (aqueous, gel, or ointment), or a solid form containing an amount of active compound, which is prepared so that it is suitable for administration to a mammal, such as a human or other animal, directly or after reconstitution. If needed, the formulation may contain pharmaceutically acceptable carriers, buffers, tonicity agents, surfactants and/or additives, as well as additional active agents where co-treatment or combination treatment may be effective.

As used herein, the terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying dis9rder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, a “therapeutically effective amount” or “effective amount” refers to an amount of the active compound that is sufficient to achieve the stated effect. A therapeutically effective amount to treat a condition in a mammal, such as a human, such as diabetes or hypertension, is an amount of active compound capable of achieving a clinically—relevant end-point in a patient or patient population, such as reduced blood glucose levels in diabetes or reduced blood pressure in hypertension or reduced cholesterol in hypertriglyceridemia or hypercholesterolemia. As non-limiting examples, administration of an effective amount of a formulation comprising an IMG-1 composition has been shown in animal studies to lower blood glucose to less than 200 mg/dL (in a diabetes animal model); lower blood pressure to less than 140/90 mmHg; and lower total cholesterol to less than 200 mg/dL.

As used herein, the term “sequence identity” refers to the identity between two or more amino acid sequences expressed in terms of the identity or similarity between the sequences. Sequence identity can be measured in terms of percentage identity; the higher the percentage, the more identical the sequences are. The percentage identity is calculated over the entire length of the sequence. Homologs or orthologs of amino acid sequences possess a relatively high degree of sequence identity when aligned using standard methods. This homology is more significant when the orthologous proteins are derived from species which are more closely related (e.g., human and mouse sequences), compared to species more distantly related (e.g., human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Nat. Acad Sci. USA 85:2444, 1988; Higgins & Sharp, Gene, 73:23744, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth Mol. Bio. 24:307-31, 1994. Altschul et al., J. Mol. Biol. 215:403-10, 1990, presents a detailed consideration of sequence alignment methods and homology calculations. The level of sequence identity may be determined using the NCBI Basic Local Alignment Search Tool (BLAST) (Altschul et al., J. Mol. Biol. 215:403-10, 1990), which is available from several sources, including the National Center for Biological Information (NCBI, National Library of Medicine, Building 38A, Room 8N805, Bethesda, Md. 20894, US) and on the Internet.

The invention includes proteins and peptides having an identity of at least 75%, at least 80%, at least 85%, at least 90%, at least 95% with a protein or peptide of the disclosure, e.g. 96% or more, 97% or more, 98% or more or 99% or more; such proteins may have the activity of the corresponding protein or peptide of the disclosure.

The present invention provides a pharmaceutical composition comprising a protein, polypeptide peptide fragment, or analogue (whether natural or synthetic) corresponding to one or more of SEQ ID NO 1, SEQ ID NO.: 2, SEQ ID NO: 3, SEQ ID NO: 4, or active regions thereof, in combination with one or more of a pharmaceutically acceptable carrier, diluent or excipient or additional active agent. The pharmaceutical composition may be formulated for oral (including sublingual), parenteral (for example subcutaneous, intramuscular, or intravenous), and/or dermal/topical including transdermal, intranasal and inhalation administration.

The invention provides formulations comprising an active of the disclosure (e.g. a protein, polypeptide peptide fragment, or analogue disclosed herein) formulated for pharmaceutical use and optionally further comprising a pharmaceutically acceptable diluent, excipient and/or carrier. The invention therefore includes pharmaceutical formulations which may include, in addition to at least one active ingredient, a pharmaceutically acceptable diluent, excipient and/or carrier. Such formulations may be used in the methods of the disclosure. Additionally, or alternatively, pharmaceutical formulations may include a buffer, stabiliser and/or other material well known to those skilled in the art. Such materials should be non-toxic and should not interfere with the efficacy of the active ingredient. The precise nature of the carrier or other material will depend on the route of administration, which may be any suitable route. The route of administration may be oral. The route of administration may be a parenteral route and particularly by infusion or injection (with or without a needle). The route of administration may be subcutaneous injection. The route of administration may be intravenous injection or infusion.

Also provided is a method of preventing, delaying the onset of, or reducing the severity of one or more conditions in a mammal associated with or caused by dysfunctional endothelial cell activity.

The amount, range, or dosage of a compound of a specific formulation, which is required to achieve the desired biological effect, will depend on several factors such as the use for which it is intended, the route of administration, the subject, and the recipient (weight, age (adult or pediatric), gender). The dose may be administered to a subject as a single unit dose, as several separate unit doses, or as a continuous infusion/absorption. The composition may be suitable for co-administration with other compounds or pharmaceutical products such as anti-hypertensive or cholesterol lowering agents.

The present invention and disclosure also include the subject matter of the following numbered clauses:

1. A method for treating a disease in a mammal characterized by endothelial dysfunction, comprising administering to a subject in need thereof of a therapeutically effective amount of a composition comprising a protein or active polypeptide according to one or more of SEQ ID NO: 1-4, or active fragment or analogue thereof.

2. The method of clause 1, wherein the disease is selected from the group consisting of cardiovascular disease, such as atherosclerosis, diabetes, including diabetes type I and diabetes type II, obesity, hypertension, including pulmonary arterial hypertension, hypercholesterolemia, and hyperlipidemia.

3. A method for modulating endothelial cell growth, the method comprising administering an effective amount of a composition comprising a polypeptide according to SEQ ID NO: 1-4, or active fragment or analogue thereof.

4. The method of any of clauses 1-3 further comprising co-administration of an active agent selected from a formulation for the treatment of one or more of diabetes, hyperglycemia, hypertension, hypercholesterolemia, metabolic disorder, and/or obesity.

5. A pharmaceutical formulation comprising a therapeutically effective amount of a protein or polypeptide comprising an amino acid sequence according to SEQ ID NO: 1-4, or active fragment, or analogue thereof, and one or more of a pharmaceutical excipient, carrier, tonicity agent, surfactant, or buffer.

6. A composition comprising a protein or active polypeptide comprising an amino acid sequence according to SEQ ID NO: 1-4, or active fragment, or analogue thereof.

The present invention is also described through examples and experimental results, which are intended as illustrative and not exhaustive, and shall be understood as not being limited thereto.

### EXAMPLES

### Example 1

A full-length polypeptide corresponding to SEQ ID NO: 01 was digested with Hydroxylamine (NH₂OH). Hydroxylamine cleaves the full-length protein at amino acid 134 at Asn in position P1 and Gly in position P1′ into two subunits; a 14 kDa fragment and a 17 kDa fragment. The resulting 14 kDa and 17 kDa fragments were resolved on a non-denaturing polyacrylamide gel, visualized and then electro eluted from the gel. The fragments were then added to Human Dermal Microvascular Endothelial Cells (CADMEC) cultures (available from Cell Applications, Inc). CADMECs were cultured in a 96-well dish at a concentration of 5,000 cells/well, according to manufacturer's instructions. Following incubation for 72 hrs the cells were subjected to an MTT cell proliferation assay (Sigma Aldrich Cell Proliferation Kit). The Cell Proliferation Kit I (MTT) is a colorimetric assay for the quantification of cellular proliferation, viability, and cytotoxicity. The assay is based on the cleavage of the tetrazolium salt MTT (3-(4,5-dimethylthiazol-2-yl)-2,5-diphenyltetrazolium bromide) in the presence of an electron-coupling reagent. Cells are then detected with the proliferation reagent, which is converted in live cells from the yellow tetrazole MTT to the purple formazan form by a cellular reductase killing the cells in the process. After solubilization, the formazan dye is quantitated using a scanning multi-well spectrophotometer (ELISA reader). The measured absorbance directly correlates to the number of viable cells; hence an increase in optical density (OD) signifies an increase in cell number.

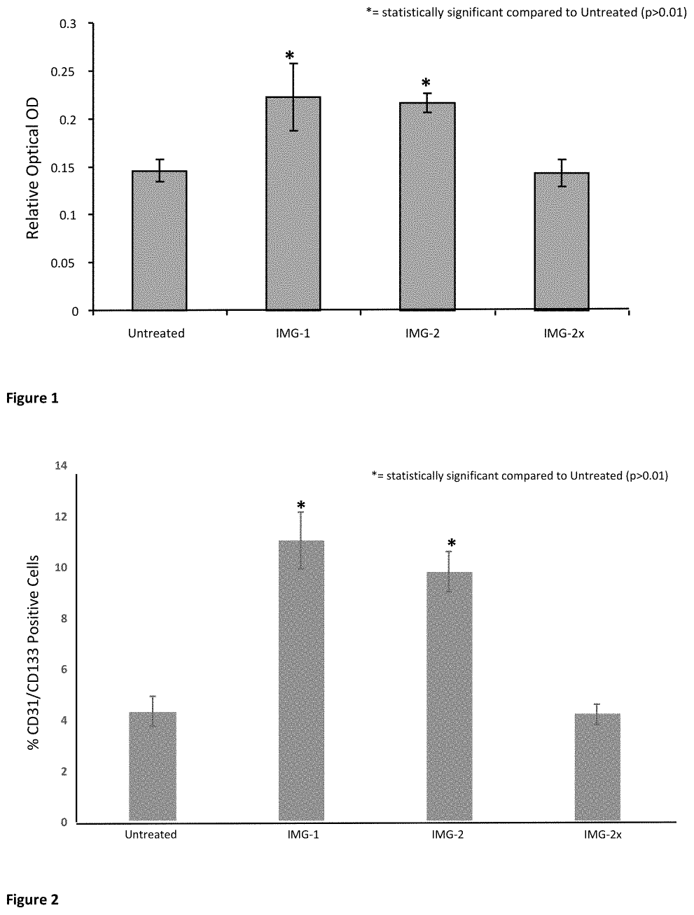

CADMECs treated with either the full length peptide or the 17 kDa fragment(s) showed a marked increase (164% and 157% respectively) in the number of endothelial cells over controls, while the cells treated with the 14 kDa fragment showed no increase in cell numbers (results shown in FIG. 1). Furthermore, treatment with formulations comprising SEQ ID NO 1 and SEQ ID NO 2 as active agent were shown to increase the number of endothelial progenitor cells compared to controls; formulations comprising SEQ ID Nos 3 or 4 are expected to show similar results given their shared sequence and functional regions.

CADMEC cells were treated with 0.5 ug/mL formulations comprising one of: full-length peptide, 14 kDa fragment, 17 kDa fragment, and allowed to grow for 72 hrs. Following 72 hours incubation, the cells were stained for CD133 and CD31 and analyzed via fluorescence-activated cell sorting (FACS) analysis. Cells that were CD31/CD133 were considered to be endothelial progenitor cells. Both cell culture samples treated with full length peptide and the 17 kDa fragment showed an increased the number of endothelial progenitor cells compared to controls (11% and 9.8% compared to 4.3%), as shown in FIG. 2, and below in Table 2.

| | Endothelial Progenitor |
|---|---|
| | Cell (CD31/CD133 |
| Peptide | double positive) |
| IMG-1 (SEQ ID NO 1) | 11% |
| IMG-2 (17 kDa fragment) | 9.8% |
| (SEQ ID NO 2) | |
| IMG-2x (14 kDa fragment) | 3.4% |
| Control | 4.4% |

### Example 2

The Zucker Diabetic Fatty {ZDF) strain of rat is widely known and commonly used to study Type 2 Diabetes associated with obesity, as well as hypertension and high cholesterol. The ZDF strain is an inbred rat model of early-onset diabetes in which all of the fa/fa male rats develop diabetes at 10 to 12 weeks of age when fed a special diet of Purina 5008. Zucker Diabetic Fatty (ZDF) rats were fed a Purina 5008 to increase their body weight. Prior to the study the animals blood glucose levels were assessed; only rats who had blood glucose levels greater than 200 mg/dL were used in the study. The animals were randomly divided into groups, untreated, 10 μg once-daily (full length peptide) intravenously (IV) and once-daily 200 μg (full length peptide) orally administered (PO). Doses were in a range of approximately: 20 μg/kg/day to 33.5 μg/kg/day (IV), and 401 μg/kg/day to 670 μg/kg/day (PO). The animals were maintained on this diet for 35 days with their weight measured twice a week. After 35 days the animals were sacrificed.

The effect of treatment with IMG-1 on diabetes-associated cardiac complications and hypertension were assayed. Fasting blood glucose (FBG) levels were assessed in the rats throughout, as well as three hours post feeding and after test article administration using a hand-held glucometer. The ZDF rats treated with full-length peptide showed a marked decrease in blood glucose levels as early as 3 days post treatment regardless of the modality administered, with all treated animals having normal FBG levels (levels below 200 mg/dL) by day 7. The blood pressure of the ZDF rats was monitored twice weekly using a CODA Noninvasive Blood Pressure Tail Cuff System blood pressure monitor.

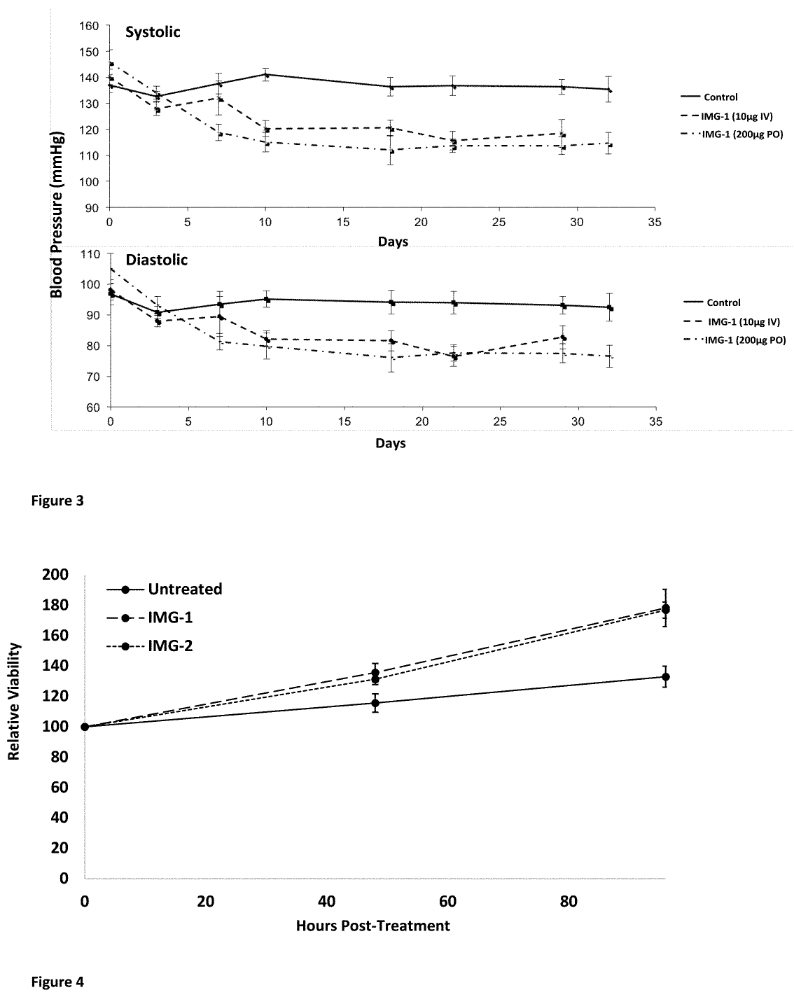

FIG. 3 shows systolic and diastolic blood pressure levels in animals treated with the full-length peptide versus those control animals. It was shown that treatment with full length peptide (whether oral or intravenous) normalized blood pressure in the diabetic animal model. Within 3 days of treatment rats treated with full-length peptide exhibited a lower blood pressure and ZDF rats who received full length peptide orally had achieved normal BP by day 7, while those who received intravenous full-length peptide had achieved normal BP by day 10 (120/82 mmHg and 115/80 mmHg for IV and PO administered full length peptide, respectively). Whereas, compared to the treated animals, the control (non-treated) ZDF rats had and maintained hypertension (>140/90 mmHg).

### Example 3

Another study was carried out that showed treatment with IMG-1 and IMG-2 impairs and/or decreases growth of Bcl-2-positive pulmonary hypertension endothelial cells.

Endothelial cells were isolated from patients undergoing Swan-Ganz (pulmonary artery endothelial cells (PAECs))—right heart catheterization (RHC) using a novel technique (J Heart Lung Transplant. 2013 July; 32(7):746-9). The isolated endothelial cells are cultured in Vascular Cell Basal Medium supplemented with the Endothelial Cell Growth Kit-BBE (available from ATCC). Swan-Ganz catheterization is the passing of a thin tube (catheter) into the right side of the heart and the arteries leading to the lungs. It is done to monitor the heart's function and blood flow and pressures in and around the heart. It has been shown that PAECs can be obtained from RHC from patients with pulmonary arterial hypertension (PAH; World Health Organization group 1).

Pulmonary arterial hypertension is a progressive disease characterized by lung endothelial cell dysfunction, vascular remodeling, small vessel loss and obstructive vasculopathy that leads to increased pulmonary vascular resistance, subsequent right heart failure, and premature death. PAECS isolated from PAH patients are hyper-proliferative, apoptosis-resistant and express, among other markers, Bcl-2. Bcl-2, (short for B-cell lymphoma 2), is a member of the Bcl family of regulator proteins that regulate cell death (apoptosis), Bcl-2 is localized to the outer membrane of the mitochondria, where it plays an important role in promoting cellular survival and inhibiting the actions of pro-apoptotic proteins.

When cells from patients undergoing RHC were isolated and treated with the compositions comprising IMG-1 or IMG-2 as active agent (corresponding to SEQ ID Nos 1 and 2, respectively), it was discovered that Bcl-2 low (non-PAH patients) cells had an increase in cell growth by over 40%; while cells that were from Bcl-2 positive PAH patients, treatment with the IMG-1 and IMG-2 peptides was shown to decrease Bcl-2 positive cell viability by 50%.

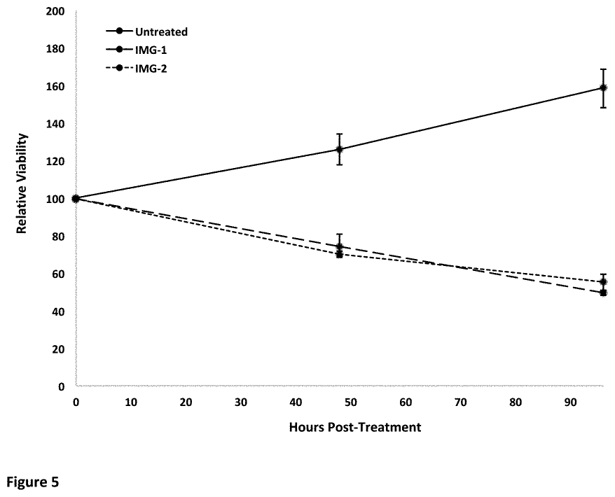

FIG. 4 shows that compositions comprising the peptides disclosed herein facilitate growth of Bcl-2 negative pulmonary hypertension endothelial cells and FIG. 5 shows that compositions comprising the peptides disclosed herein facilitate impairment in the growth of Bcl-2 positive pulmonary hypertension endothelial cells, supporting the use of the peptides as an anti-hypertensive agent and for the treatment of pulmonary arterial hypertension.

PAECs from non-PAH patients (Bcl-2 negative, as confirmed by western blot analysis) and treated IMG-1 and IMG-2 showed a marked increase in cell growth, with a relative increase in cell number averaging a growth of 35% and 31% (IMG-1 and IMG-2 respectively) vs 15.6% cell growth in controls at 48 hrs and 78% and 77% vs 33% at 96 hrs post treatment. Demonstrating an overall increase of ˜40% cell growth over a 96 hr time period (FIG. 4). While PAECs from PAH patients (Bcl-2 positive, as confirmed by western blot analysis) had a significant decrease in cell growth with relative cell numbers equivalent to −26% and −30% of their starting values at 48 hrs (IMG-1 and IMG-2 respectively) while controls demonstrated a 26% increase. By 96 hrs, the IMG-1 and IMG-2 treated cells demonstrated relative cells numbers as low as −51% (IMG-1) and −45% (IMG-2) compared to an increase of 58% in cell number in the control, denoting a significant decrease in proliferation between treated and control PAECs (FIG. 5).

The compositions and methods described herein can include, or be used in combination with, other agents or therapeutic modalities. In one embodiment, the methods described herein include administering to a subject a combination comprising a peptide molecule as described herein, in combination with an agent or therapeutic procedure or modality, in an amount effective to treat or prevent a disorder. The peptide molecule and the agent or therapeutic procedure or modality can be administered simultaneously or sequentially in any order. Any combination and sequence of the peptide molecules and other therapeutic agents, procedures or modalities (e.g., as described herein) can be used. The peptide molecule and/or other therapeutic agents, procedures or modalities can be administered during periods of active disorder, or less active disease state. The peptide molecule can be administered before the other treatment, concurrently with the treatment, or post-treatment.

Formulations, peptide, peptide fragments and compositions of the present invention may be referred to by nomenclature in graphs and figures (such as “IMG-1” or “IMG-2”) simply to distinguish treated subjects from controls or untreated samples. Nomenclature is not intended to be limiting in any manner.

Experiments disclosed herein were, in part, conducted in murine models, and cell culture experiments utilized mammalian (human) cells. Using allometric scaling, it is possible to predict suitable and exemplary dosage ranges for the administration of compositions comprising polypeptide active agent, as disclosed herein, to adult humans. Allometric scaling is an empirical approach, where the exchange of drug dose is based on normalization of dose to body surface area and is well characterized and understood in the art. This approach assumes that there are some unique characteristics on anatomical, physiological, and biochemical process among species, and the possible difference in pharmacokinetics/physiological time is, as such, accounted for by allometric scaling. This method is frequently used in research for experimental purpose to predict an approximate dose on the basis of data existing in other species. The dose by factor method is an empirical approach and uses the no observed adverse effect levels (NOAEL) of drug from preclinical toxicological studies to estimate human equivalent dose (HED). The dose by factor method applies an exponent for body surface area (0.67), which account for difference in metabolic rate, to convert doses between animals and humans. Thus, HED is determined by the equation:

HED (mg/kg=Animal NOAEL mg/kg)×(Weight animal [kg]/Weight human [kg])(1-0.67) Eq. (1)

Based on an exemplary formulation as disclosed herein, the NOAEL value in a rat weighing approximately 300 g is between 300 μg/kg-1700 μg/kg (twice daily).

To calculate the starting dose range for human studies:

HED μg/kg=300×(0.30/60)(0.33)=52.21 μg/kg and up to 295.86 μg/kg (twice daily). Thus, for a 60 kg human, an exemplary dose would be 3.133 g up to 17.753 g, twice daily.

Throughout the description and claims of this specification, the words “comprise” and “contain” and variations of them mean “including but not limited to”, and they are not intended to (and do not) exclude other moieties, additives, components, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The present disclosure should not be construed to limit the invention, as modifications and variations in the embodiments disclosed herein may be made by those of ordinary skill in the art without departing from the scope and spirit of the present invention. All publications and other references cited in this application are hereby incorporated by reference in their entirety.

1. A polypeptide or formulation comprising a polypeptide for use in the treatment of a disease characterized by endothelial dysfunction, wherein the polypeptide comprises an amino acid sequence having at least 75% sequence identity to the amino acid sequence set forth in SEQ ID NO: 1, 2, 3, or 4.

2. The polypeptide or formulation of claim 1, wherein the disease characterized by endothelial dysfunction is selected from cardiovascular disease, atherosclerosis, obesity and pulmonary arterial hypertension.

3. The polypeptide or formulation of claim 1, wherein the polypeptide has at least 80%, 85%, 90%, 95%, 98% or 99% sequence identity to the amino acid sequence set forth in SEQ ID NO: 1, 2, 3, or 4.

4. The polypeptide or formulation of claim 1, wherein the polypeptide comprises the amino acid sequence set forth in SEQ ID NO: 1, 2, 3, or 4.

5. The polypeptide or formulation of claim 1, wherein the polypeptide consists of the amino acid sequence set forth in SEQ ID NO: 1, 2, 3, or 4.

6. The formulation of claim 1, wherein the formulation is an oral pharmaceutical formulation.

7. The formulation of claim 1, wherein the formulation is a parenteral pharmaceutical formulation.

8. The formulation of claim 1, wherein the formulation comprises one or more pharmaceutically acceptable carrier and/or one or more pharmaceutically acceptable diluent and/or one or more pharmaceutically acceptable excipient.

9. The formulation of claim 1, wherein the formulation is an aqueous pharmaceutical formulation.

10. The formulation of claim 9, wherein the polypeptide is present at a concentration of 0.05 to 5 μg/L.

11. The formulation of claim 9, wherein the polypeptide is present at a concentration of 0.1 to 1 μg/L.

12. The formulation of any of claims 9 to 11, wherein the aqueous pharmaceutical formulation comprises a buffer, optionally a buffer having a physiological pH.

13. The formulation of claim 12, wherein the buffer is phosphate buffered saline.

14. A method for modulating endothelial cell growth, the method comprising contacting an endothelial cell with a polypeptide or formulation comprising a polypeptide,
wherein the polypeptide comprises a polypeptide comprising an amino acid sequence having at least 75% sequence identity to the amino acid sequence set forth in SEQ ID NO: 1, 2, 3, or 4.

15. The method of claim 14, wherein the contacting is performed in an aqueous solution.

16. (canceled)

17. Use of a polypeptide or formulation comprising a polypeptide for modulating endothelial cell growth,
wherein the polypeptide comprises a polypeptide comprising an amino acid sequence having at least 75% sequence identity to the amino acid sequence set forth in SEQ ID NO: 1, 2, 3, or 4.

18. (canceled)

19. A method for treating a disease characterized by endothelial dysfunction, comprising administering to a subject in need thereof of a therapeutically effective amount of a composition comprising a polypeptide comprising an amino acid sequence having at least 75% sequence identity to the amino acid sequence set forth in SEQ ID NO: 1, 2, 3, or 4, or an active fragment or analogue thereof.

20. The method of claim 19, wherein the disease characterized by endothelial dysfunction is selected from cardiovascular disease, atherosclerosis, obesity and pulmonary arterial hypertension.

21. The method of claim 19, wherein the polypeptide has at least 80%, 85%, 90%, 95%, 98% or 99% sequence identity to the amino acid sequence set forth in SEQ ID NO: 1, 2, 3, or 4.

22. The method of claim 19, wherein the polypeptide comprises the amino acid sequence set forth in SEQ ID NO: 1, 2, 3, or 4.

23. The method of claim 19, wherein the polypeptide consists of the amino acid sequence set forth in SEQ ID NO: 1, 2, 3, or 4.

24-28. (canceled)
